(19) United States
(12) Patent Application Publication
(10) Pub. No.: US 2014/0100564 A1
(43) Pub. Date: Apr. 10, 2014

(54) JAW ASSEMBLIES FOR ELECTROSURGICAL INSTRUMENTS AND METHODS OF MANUFACTURING JAW ASSEMBLIES

(71) Applicant: COVIDIEN LP, MANSFIELD, MA (US)

(72) Inventor: DAVID M. GARRISON, LONGMONT, CO (US)

(73) Assignee: COVIDIEN LP, MANSFIELD, MA (US)

(21) Appl. No.: 14/019,094

(22) Filed: Sep. 5, 2013

Related U.S. Application Data
(60) Provisional application No. 61/711,071, filed on Oct. 8, 2012.

(51) Int. Cl. A61B 18/08

(52) U.S. Cl. CPC A61B 18/085
USPC 606/41; 156/60

(57) ABSTRACT

A jaw assembly includes a jaw member and a structural insert. The jaw member includes an arm member and a support base extending distally from the arm member. The arm member defines a first portion of the jaw member. The support base defines a second portion and a third portion of the jaw member. The second portion defines a cavity disposed between the first portion and the third portion. At least a portion of the structural insert is disposed within the cavity.

### CROSS REFERENCE TO RELATED APPLICATION

The present application claims the benefit of and priority to U.S. Provisional Application Ser. No. 61/711,071, filed on Oct. 8, 2012, the entire contents of which are incorporated herein by reference.

### BACKGROUND

1. Technical Field

The present disclosure relates to electrosurgical instruments. More particularly, the present disclosure relates to jaw assemblies for use in electrosurgical instruments and methods of manufacturing jaw assemblies.

2. Discussion of Related Art

Electrosurgical instruments have become widely used by surgeons. Electrosurgery involves the application of thermal and/or electrical energy to cut, dissect, ablate, coagulate, cauterize, seal or otherwise treat biological tissue during a surgical procedure. Electrosurgery is typically performed using an electrosurgical generator operable to output energy and a handpiece including a surgical instrument (e.g., end effector) adapted to transmit energy to a tissue site during electrosurgical procedures. Electrosurgery can be performed using either a monopolar or a bipolar instrument.

The basic purpose of both monopolar and bipolar electrosurgery is to produce heat to achieve the desired tissue/clinical effect. In monopolar electrosurgery, devices use an instrument with a single, active electrode to deliver energy from an electrosurgical generator to tissue, and a patient return electrode or pad that is attached externally to the patient (e.g., a plate positioned on the patient's thigh or back) as the means to complete the electrical circuit between the electrosurgical generator and the patient. When the electrosurgical energy is applied, the energy travels from the active electrode, to the surgical site, through the patient and to the return electrode. In bipolar electrosurgery, both the active electrode and return electrode functions are performed at the site of surgery. Bipolar electrosurgical devices include two electrodes that are located in proximity to one another for the application of current between their surfaces. Bipolar electrosurgical current travels from one electrode, through the intervening tissue to the other electrode to complete the electrical circuit. Bipolar instruments generally include end-effectors, such as grippers, cutters, forceps, dissectors and the like.

Forceps utilize mechanical action to constrict, grasp, dissect and/or clamp tissue. By utilizing an electrosurgical forceps, a surgeon can utilize both mechanical clamping action and electrosurgical energy to effect hemostasis by heating the tissue and blood vessels to cauterize, coagulate/desiccate, seal and/or divide tissue. Bipolar electrosurgical forceps utilize two generally opposing electrodes that are operably associated with the inner opposing surfaces of end effectors and that are both electrically coupled to an electrosurgical generator. In bipolar forceps, the end-effector assembly generally includes opposing jaw assemblies pivotably mounted with respect to one another. In bipolar configuration, only the tissue grasped between the jaw assemblies is included in the electrical circuit. Because the return function is performed by one jaw assembly of the forceps, no patient return electrode is needed.

By utilizing an electrosurgical forceps, a surgeon can cauterize, coagulate/desiccate and/or seal tissue and/or simply reduce or slow bleeding by controlling the intensity, frequency and duration of the electrosurgical energy applied through the jaw assemblies to the tissue. During the sealing process, mechanical factors such as the pressure applied between opposing jaw assemblies and the gap distance between the electrically-conductive tissue-contacting surfaces (electrodes) of the jaw assemblies play a role in determining the resulting thickness of the sealed tissue and effectiveness of the seal.

A variety of types of end-effector assemblies have been employed for various types of electrosurgery using a variety of types of monopolar and bipolar electrosurgical instruments. Jaw assembly components of end-effector assemblies for use in electrosurgical instruments are required to meet specific tolerance requirements for proper jaw alignment and other closely-toleranced features, and are generally manufactured by expensive and time-consuming processes that typically involve complex machining operations. Gap tolerances and/or surface parallelism and flatness tolerances are parameters that, if properly controlled, can contribute to a consistent and effective tissue seal. Thermal resistance, strength and rigidity of surgical jaw assemblies also play a role in determining the reliability and effectiveness of electrosurgical instruments.

### SUMMARY

A continuing need exists for tightly-toleranced jaw assembly components that can be readily integrated into manufacturing assembly processes for the production of end-effector assemblies for use in electrosurgical instruments, such as electrosurgical forceps. Further need exists for the development of a manufacturing process that effectively fabricates jaw assembly components at low cost, and results in the formation of a reliable electrosurgical instrument that meets specific tolerance requirements for proper jaw alignment and other tightly-toleranced jaw assembly features, with reduction or elimination of complex machining operations. A continuing need exists for improved thermal resistance, strength and rigidity of jaw assemblies.

According to an aspect of the present disclosure, a jaw assembly is provided. The jaw assembly includes a jaw member and a structural insert. The jaw member includes an arm member and a support base extending distally from the arm member. The arm member defines a first portion of the jaw member. The support base defines a second portion and a third portion of the jaw member. The second portion defines a cavity disposed between the first portion and the third portion. At least a portion of the structural insert is disposed within the cavity.

According to another aspect of the present disclosure, an end-effector assembly is provided. The end-effector assembly includes opposing first and second jaw assemblies pivotably mounted with respect to one another. The first jaw assembly includes a first jaw member including a first arm member, a first support base extending distally from the first arm member, and a structural insert. The first arm member defines one or more apertures at least partially therethrough and defines a first portion of the first jaw member. The first support base defines a second portion and a third portion of the first jaw member, wherein the second portion defines a cavity disposed between the first portion and the third portion. At least a portion of the structural insert is disposed within the cavity. The second jaw assembly includes a second jaw member including a second arm member defining one or more apertures at least partially therethrough and a second support base extending distally from the second arm member. The first jaw assembly further includes a first electrically-conductive tissue-engaging structure and a first non-electrically conductive member. The first non-electrically conductive member is configured to electrically isolate the electrically-conductive tissue-engaging structure from the first support base of the first jaw member. The second jaw assembly further includes a second electrically-conductive tissue-engaging structure and a second non-electrically conductive member. The second non-electrically conductive member is configured to electrically isolate the electrically-conductive tissue-engaging structure from the second support base of the second jaw member. One or more pivot pins are engaged with the one or more apertures of the first and second jaw members such that the first and second jaw assemblies are pivotably mounted with respect to one another.

According to another aspect of the present disclosure, a method of manufacturing a jaw assembly is provided. The method includes the initial steps of providing an electrically-conductive tissue-engaging structure, providing a structural insert, and providing a jaw member including a support base extending distally from an arm member. The arm member defines a first portion of the jaw member. The support base defines a second portion and a third portion of the jaw member. The second portion defines a cavity disposed between the first portion and the third portion and configured to receive at least a portion of the structural insert therein. The method also includes the steps of performing a first bonding process to join the structural insert and the jaw member, providing a non-electrically conductive member configured to electrically isolate the electrically-conductive tissue-engaging structure from the jaw member, and performing a second bonding process to join the electrically-conductive tissue-engaging structure, non-electrically conductive member and the jaw member, thereby forming a jaw assembly.

### BRIEF DESCRIPTION OF THE DRAWINGS

Objects and features of the presently-disclosed jaw assemblies for use in electrosurgical instruments and methods of manufacturing jaw assemblies will become apparent to those of ordinary skill in the art when descriptions of various embodiments thereof are read with reference to the accompanying drawings, of which:

FIG. 1 is a right, side view of an endoscopic bipolar forceps showing a housing, a rotatable member, a shaft and an end-effector assembly in accordance with an embodiment of the present disclosure;

FIG. 2 is a schematic diagram of a jaw assembly of an end-effector assembly, such as the end-effector assembly of the forceps shown in FIG. 1, in accordance with an embodiment of the present disclosure;

FIG. 3 is a schematic diagram of an electrical connector portion of a jaw assembly, such as the jaw assembly shown in FIG. 2, in accordance with an embodiment of the present disclosure;

FIG. 4 is a schematic diagram of another embodiment of an electrical connector portion of a jaw assembly, such as the jaw assembly shown in FIG. 2, in accordance with the present disclosure;

FIG. 5 is a schematic diagram of a portion of a jaw assembly, with parts separated, in accordance with an embodiment of the present disclosure;

FIG. 6 is a schematic diagram of a jaw assembly of an end-effector assembly, such as the end-effector assembly of the forceps shown in FIG. 1, in accordance with an embodiment of the present disclosure;

FIG. 7 is an enlarged, cross-sectional view taken along the section lines 7-7 of FIG. 6;

FIG. 8 is an enlarged, end view of an end-effector assembly including the jaw assembly shown in FIG. 6 in accordance with an embodiment of the present disclosure;

FIG. 9 is a schematic diagram of a portion of a jaw assembly in accordance with an embodiment of the present disclosure;

FIG. 10 is an enlarged, cross-sectional view of a jaw assembly, such as the jaw assembly shown in FIG. 11, in accordance with an embodiment of the present disclosure;

FIG. 11 is a schematic diagram of a portion of a jaw assembly in accordance with an embodiment of the present disclosure;

FIG. 12 is a schematic diagram of a portion of the jaw assembly shown in

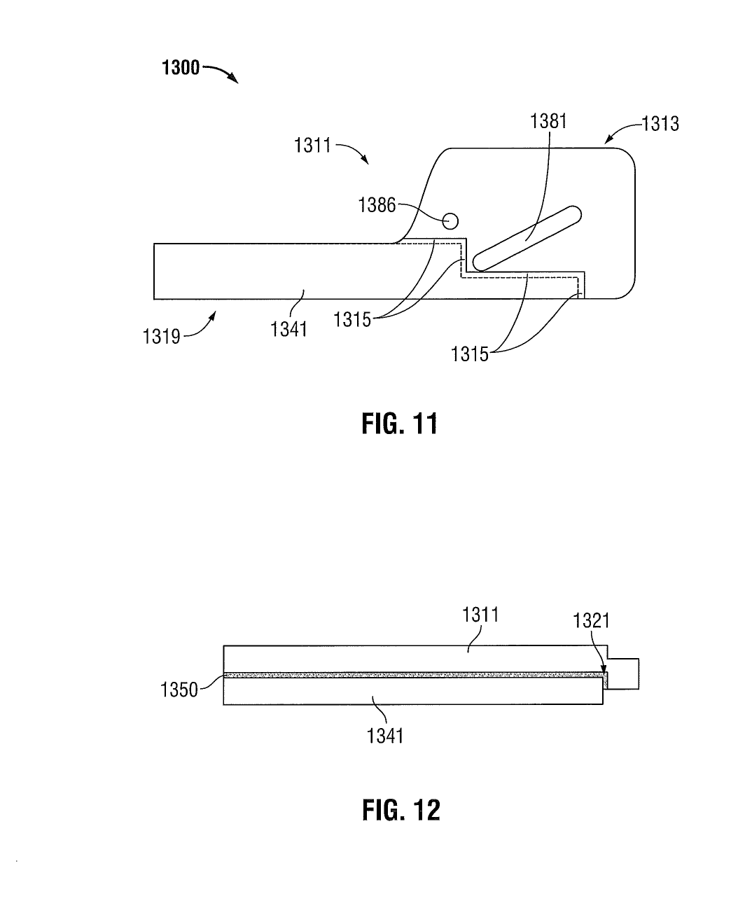

FIG. 11 in accordance with an embodiment of the present disclosure;

FIG. 13 is an enlarged, cross-sectional view of a portion of an end-effector assembly in accordance with an embodiment of the present disclosure;

FIG. 14 is an enlarged, cross-sectional view of the area of detail indicated in FIG. 13 illustrating a knife channel of the end-effector assembly in accordance with an embodiment of the present disclosure; and

FIG. 15 is a flowchart illustrating a method of manufacturing a jaw assembly in accordance with an embodiment of the present disclosure.

### DETAILED DESCRIPTION

Hereinafter, embodiments of jaw assemblies for use in electrosurgical instruments and methods of manufacturing jaw assemblies of the present disclosure are described with reference to the accompanying drawings. Like reference numerals may refer to similar or identical elements throughout the description of the figures. As shown in the drawings and as used in this description, and as is traditional when referring to relative positioning on an object, the term “proximal” refers to that portion of the apparatus, or component thereof, closer to the user and the term “distal” refers to that portion of the apparatus, or component thereof, farther from the user.

This description may use the phrases “in an embodiment,” “in embodiments,” “in some embodiments,” or “in other embodiments,” which may each refer to one or more of the same or different embodiments in accordance with the present disclosure.

Various embodiments of the present disclosure provide electrosurgical instruments suitable for sealing, cauterizing, coagulating/desiccating and/or cutting vessels and vascular tissue. Various embodiments of the present disclosure provide an electrosurgical forceps with an end-effector assembly including opposing jaw assemblies pivotably mounted with respect to one another. Various embodiments of the present disclosure provide jaw assemblies including one or more structural inserts and formed to meet specific tolerance requirements for proper jaw alignment, thermal resistance, strength and rigidity. Various embodiments of the present disclosure provide methods of manufacturing jaw assembly components of end-effector assemblies for use in electrosurgical instruments, including without limitation, bipolar forceps.

Embodiments of the presently-disclosed electrosurgical forceps may be suitable for utilization in endoscopic surgical procedures and/or suitable for utilization in open surgical applications. Embodiments of the presently-disclosed bipolar forceps may be implemented using electromagnetic radiation at microwave frequencies, radio frequencies (RF) or at other frequencies. Although the following description describes the use of an endoscopic bipolar forceps, the teachings of the present disclosure may also apply to a variety of electrosurgical devices that include jaw assemblies.

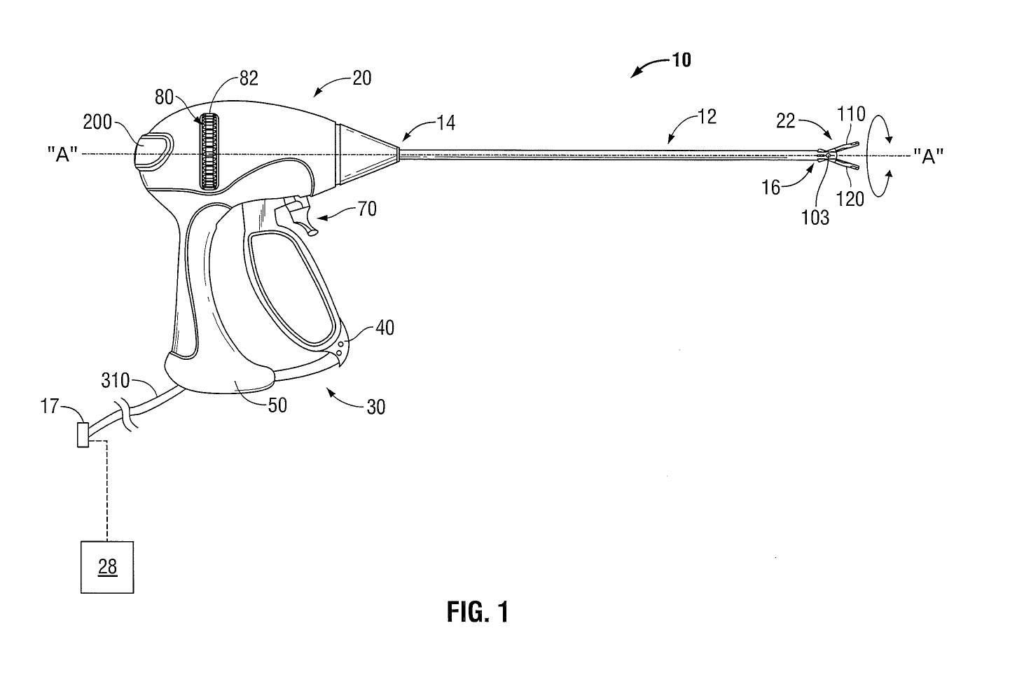

In FIG. 1, an embodiment of an endoscopic bipolar forceps 10 is shown for use with various surgical procedures and generally includes a housing 20, a handle assembly 30, a rotatable assembly 80, a trigger assembly 70 and an end-effector assembly 22 that mutually cooperate to grasp, seal and/or divide tissue, e.g., tubular vessels and vascular tissue (not shown). Although FIG. 1 depicts a bipolar forceps 10 for use in connection with endoscopic surgical procedures, the teachings of the present disclosure may also apply to more traditional open surgical procedures. For the purposes herein, the forceps 10 is described in terms of an endoscopic instrument; however, an open version of the forceps (not shown) may also include the same or similar operating components and features as described below.

Forceps 10 includes a shaft 12 having a distal end 16 configured to mechanically engage the end-effector assembly 22 and a proximal end 14 configured to mechanically engage the housing 20. In some embodiments, the shaft 12 has a length from the proximal side of the handle assembly 30 to the distal side of the forceps 10 in a range of about 7 centimeters to about 44 centimeters. End-effector assembly 22 may be selectively and releaseably engageable with the distal end 16 of the shaft 12, and/or the proximal end 14 of the shaft 12 may be selectively and releaseably engageable with the housing 20 and the handle assembly 30.

The proximal end 14 of the shaft 12 is received within the housing 20, and connections relating thereto are disclosed in commonly assigned U.S. Pat. No. 7,150,097 entitled “METHOD OF MANUFACTURING JAW ASSEMBLY FOR VESSEL SEALER AND DIVIDER”, commonly assigned U.S. Pat. No. 7,156,846 entitled “VESSEL SEALER AND DIVIDER FOR USE WITH SMALL TROCARS AND CANNULAS”, commonly assigned U.S. Pat. No. 7,597,693 entitled “VESSEL SEALER AND DIVIDER FOR USE WITH SMALL TROCARS AND CANNULAS” and commonly assigned U.S. Pat. No. 7,771,425 entitled “VESSEL SEALER AND DIVIDER HAVING A VARIABLE JAW CLAMPING MECHANISM”.

Forceps 10 includes an electrosurgical cable 310. Electrosurgical cable 310 may be formed from a suitable flexible, semi-rigid or rigid cable, and may connect directly to an electrosurgical power generating source 28. In some embodiments, the electrosurgical cable 310 connects the forceps 10 to a connector 17, which further operably connects the instrument 10 to the electrosurgical power generating source 28. Cable 310 may be internally divided into one or more cable leads each of which transmits electrosurgical energy through their respective feed paths to the end-effector assembly 22.

Electrosurgical power generating source 28 may be any generator suitable for use with electrosurgical devices, and may be configured to provide various frequencies of electromagnetic energy. Examples of electrosurgical generators that may be suitable for use as a source of electrosurgical energy are commercially available under the trademarks FORCE EZ™, FORCE FX™, and FORCE TRIAD™ offered by Covidien Surgical Solutions of Boulder, Colo. Forceps 10 may alternatively be configured as a wireless device or battery-powered.

End-effector assembly 22 generally includes a pair of opposing jaw assemblies 110 and 120 pivotably mounted with respect to one another. End-effector assembly 22 may be configured as a bilateral jaw assembly, i.e., both jaw assemblies 110 and 120 move relative to one another. Alternatively, the forceps 10 may include a unilateral assembly, i.e., the end-effector assembly 22 may include a stationary or fixed jaw assembly, e.g., 120, mounted in fixed relation to the shaft 12 and a pivoting jaw assembly, e.g., 110, mounted about a pivot pin 103 coupled to the stationary jaw assembly. Jaw assemblies 110 and 120 may be curved at various angles to facilitate manipulation of tissue and/or to provide enhanced line-of-sight for accessing targeted tissues.

As shown in FIG. 1, the end-effector assembly 22 is rotatable about a longitudinal axis “A-A” through rotation, either manually or otherwise, of the rotatable assembly 80. Rotatable assembly 80 generally includes two halves (not shown), which, when assembled about a tube of shaft 12, form a generally circular rotatable member 82. Rotatable assembly 80, or portions thereof, may be configured to house a drive assembly (not shown) and/or a knife assembly (not shown), or components thereof. A reciprocating sleeve (not shown) is slidingly disposed within the shaft 12 and remotely operable by the drive assembly (not shown). Examples of rotatable assembly embodiments, drive assembly embodiments, and knife assembly embodiments of the forceps 10 are described in the above-mentioned, commonly-assigned U.S. Pat. Nos. 7,150,097, 7,156,846, 7,597,693 and 7,771,425.

Handle assembly 30 includes a fixed handle 50 and a movable handle 40. In some embodiments, the fixed handle 50 is integrally associated with the housing 20, and the movable handle 40 is selectively movable relative to the fixed handle 50. Movable handle 40 of the handle assembly 30 is ultimately connected to the drive assembly (not shown). As can be appreciated, applying force to move the movable handle 40 toward the fixed handle 50 pulls the drive sleeve (not shown) proximally to impart movement to the jaw assemblies 110 and 120 from an open position, wherein the jaw assemblies 110 and 120 are disposed in spaced relation relative to one another, to a clamping or closed position, wherein the jaw assemblies 110 and 120 cooperate to grasp tissue therebetween. Examples of handle assembly embodiments of the forceps 10 are described in the above-mentioned, commonly-assigned U.S. Pat. Nos. 7,150,097, 7,156,846, 7,597,693 and 7,771,425.

Forceps 10 includes a switch 200 configured to permit the user to selectively activate the forceps 10 in a variety of different orientations, i.e., multi-oriented activation. As can be appreciated, this simplifies activation. When the switch 200 is depressed, electrosurgical energy is transferred through one or more electrical leads (not shown) to the jaw assemblies 110 and 120. Although FIG. 1 depicts the switch 200 disposed at the proximal end of the housing assembly 20, switch 200 may be disposed on another part of the forceps 10 (e.g., the fixed handle 50, rotatable member 82, etc.) or another location on the housing assembly 20.

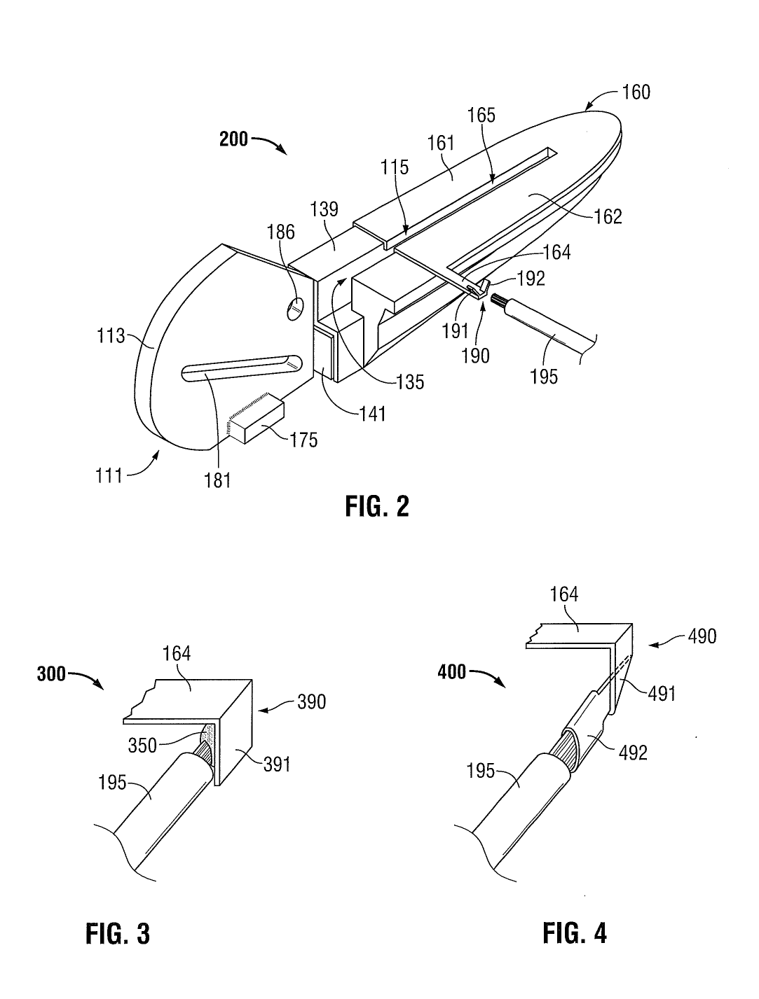

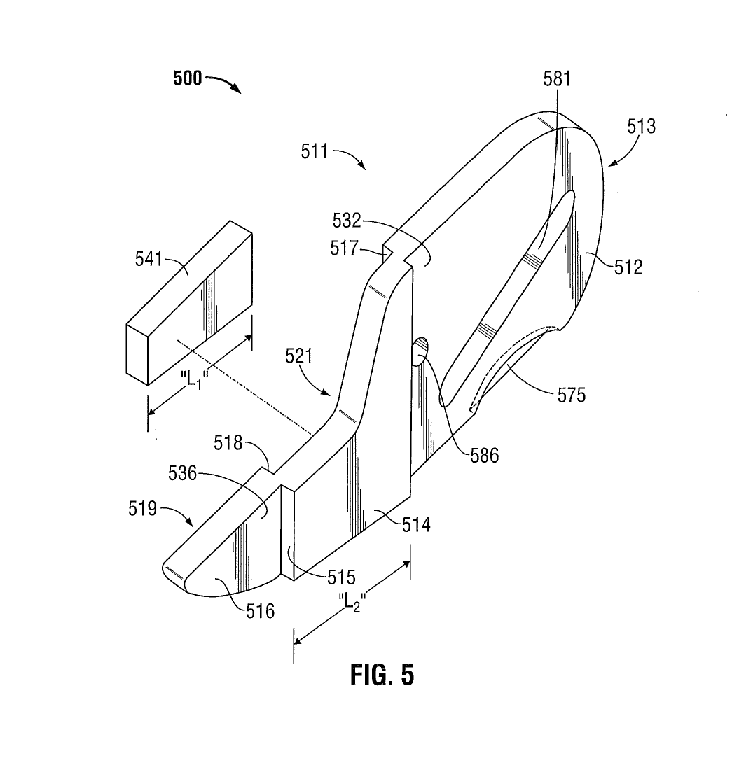

FIG. 2 shows a jaw assembly (shown generally as 200) according to an embodiment of the present disclosure that includes a jaw member 111, a structural insert 141, an electrically-conductive tissue-engaging surface or sealing plate 160, and a non-electrically conductive member 139. Jaw member 111 includes an arm member 113 and a support base (e.g., support base 519 shown in FIG. 5) that extends distally from the arm member 113. Structural insert 141 is disposed at least partially within a cavity (e.g., cavity 520 shown in FIG. 5) associated with the support base, e.g., to provide enhanced strength and/or rigidity of the jaw assembly 200. In some embodiments, the non-electrically conductive member 139 may be configured to engage the sealing plate 160 and/or the jaw member 111. As shown in FIG. 2, the sealing plate 160, the non-electrically conductive member 139 and the jaw member 111, when assembled, form a longitudinally-oriented slot or knife channel 115 defined therethrough for reciprocation of a knife blade (not shown).

Jaw member 111 may define one or more apertures at least partially therethrough, e.g., pivot holes and/or pin slots or openings. In some embodiments, as shown in FIG. 2, the arm member 113 includes an elongated angled slot 181 and a pivot hole 186 defined therethrough. Jaw member 111 may additionally, or alternatively, include one or more jaw alignment spacers, e.g., jaw alignment spacer 175, integrally formed with or otherwise coupled to the arm member 113. Jaw alignment spacer 175 may include any suitable material. In some embodiments, the jaw alignment spacer 175 may include metal, ceramic, polymeric, glass, and/or other material. Jaw alignment spacer 175 may be formed by molding stamping, machining, welding, bonding, and/or other suitable method. In some embodiments, the jaw alignment spacer 175 may be formed of the same material as the jaw member 111. The shape and size of the jaw alignment spacer 175 may be varied from the configuration depicted in FIG. 2.

Sealing plate 160 generally includes a first portion 161 and a second portion 162, and may include an electrode tip portion (not shown) for contacting tissue. First portion 161 and the second portion 162 of the sealing plate 160 are at least partially separated by a longitudinally-oriented slot or knife channel 115 defined therebetween. Sealing plate 160 may be coupled to the non-electrically conductive member 139 in any suitable manner, e.g., joined by brazing and/or adhesive bonding. The shape and size of the sealing plate 160 and the knife channel 115 may be varied from the configuration depicted in FIG. 2.

In some embodiments, as shown in FIGS. 2 through 4, the sealing plate 160 includes a connector portion (shown generally as 300 and 400 in FIGS. 3 and 4) including a first connector portion (e.g., 190 shown in FIG. 2, 390 shown in FIGS. 3, and 490 shown in FIG. 4) for connection to a wire conductor 195. First connector portion 190, 390, 490 may be mechanically coupled to the wire conductor 195 by crimping and/or solder (e.g., solder 350 shown in FIG. 3).

In some embodiments, as shown in FIG. 2, the first connector portion 190 includes two prong-like elements 191 and 192 configured to be electro-mechanically coupled to the wire conductor 195, e.g., by crimping after bending of the two prong-like elements 191 and 192. In some embodiments, as shown in FIG. 3, the first connector portion 390 includes a flange 391 configured to be electro-mechanically coupled to the wire conductor 195 by solder 350. In some embodiments, as shown in FIG. 4, the first connector portion 490 includes a flange 491 and a tubular portion 492 configured to be electro-mechanically coupled to the wire conductor 195 by crimping of the tubular portion 492. Wire conductor 195, in turn, is electrically coupled with an electrosurgical energy source (not shown). Sealing plate 160 may include a second connector portion 194 configured to electrically couple the first connector portion 190, 390, 490 to the sealing plate 160 (FIG. 2).

Non-electrically conductive member 139 is configured to electrically isolate, at least in part, the sealing plate 160 from the jaw member 111, or portion thereof (e.g., the support base). Non-electrically conductive member 139 includes a channel 135 defined therein which extends longitudinally along a portion of the non-electrically conductive member 139 and which aligns in vertical registration with the knife channel 165 defined in the sealing plate 160 to facilitate translation of a knife (not shown) therethrough. Non-electrically conductive member 139 may be formed of any suitable electrically insulative material. In some embodiments, non-electrically conductive member 139 is formed of non-electrically conductive ceramic, and may provide enhanced thermal resistance, strength, and/or rigidity of the jaw assembly 200. Non-electrically conductive member 139 may be formed by any suitable process, e.g., injection molding, ceramic injection molding (CIM), or compression molding.

Jaw assembly 200 may include additional, fewer, or different components than shown in FIG. 2, depending upon a particular purpose or to achieve a desired result. Non-electrically conductive member 139 may be used for joining together sealing plates and support bases of jaw members of varied geometries, e.g., lengths and curvatures, or having additional, fewer, or different features than the jaw member 111, such that variously-configured jaw assemblies may be fabricated and assembled into various end-effector configurations, e.g., depending upon design of specialized electrosurgical instruments.

FIG. 5 shows a portion of a jaw assembly (shown generally as 500) according to an embodiment of the present disclosure that includes a jaw member 511. Jaw member 511 may be formed by any suitable process, e.g., machining, stamping, electrical discharge machining (EDM), metal injection molding (MIM), and/or fineblanking. Jaw member 511 includes a support base 519 that extends distally from an arm member 513. Arm member 513 and the support base 519 are generally formed from metal, e.g., steel, and may include non-metal elements. Arm member 513 and the support base 519 may be formed from any suitable material or combination of materials.

In some embodiments, the arm member 513 and support base 519 are separately fabricated and each includes an engagement structure (not shown) configured for attachment to one another. Examples of engagement structure embodiments are described in commonly-assigned U.S. patent application Ser. No. 13/243,628 filed on Sep. 23, 2011, entitled “END-EFFECTOR ASSEMBLIES FOR ELECTROSURGICAL INSTRUMENTS AND METHODS OF MANUFACTURING JAW ASSEMBLY COMPONENTS OF END-EFFECTOR ASSEMBLIES”.

Arm member 513 defines a first portion 512 of the jaw member 511. Support base 519 defines a second portion 514 and a third portion 516 of the jaw member 511. In some embodiments, as shown in FIG. 5, the second portion 514 defines a cavity 521 disposed between the distal end 517 of the first portion 512 and the proximal end 518 of the third portion 516. Cavity 521 is configured to receive at least a portion of a structural insert 541 therein. Structural insert 541 may have any suitable length “L₁”.

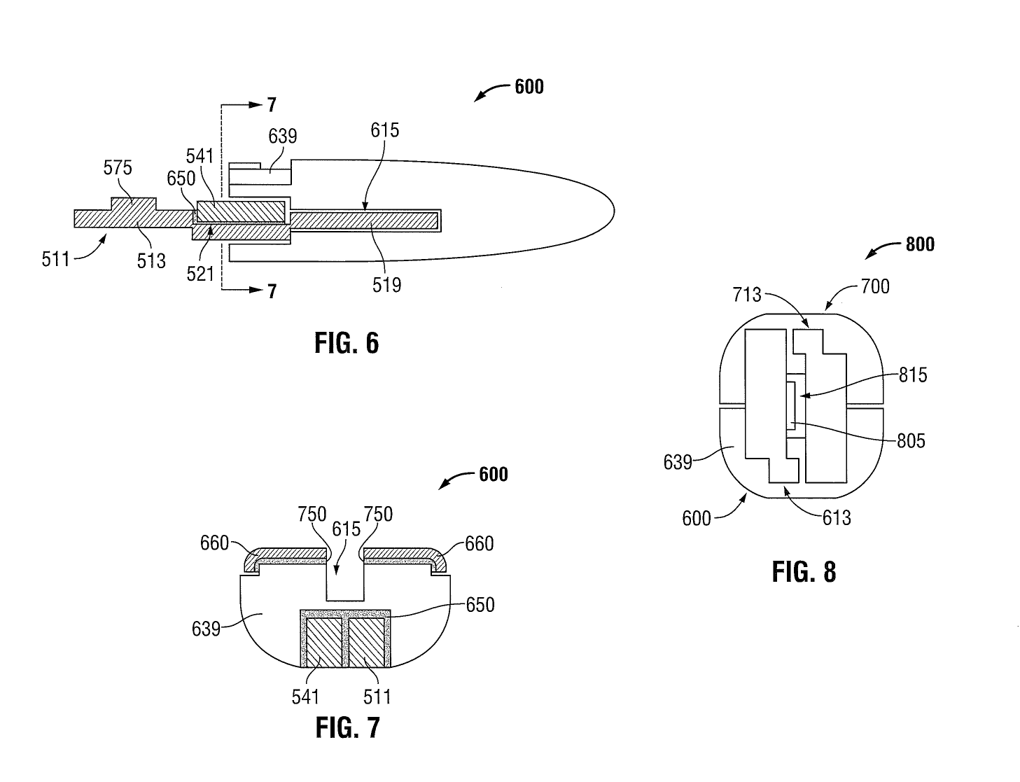

Structural insert 541 may be formed from any suitable material, and may be joined to the jaw member 511 by any suitable process, e.g., welding, brazing, soldering, and/or adhesive bonding. A bonding material 650 (shown in FIGS. 6 and 7), e.g., brazing material, adhesive material, or solder material, may be disposed within the cavity 521, or portion thereof, between the structural insert 541 and the support base 519 of the jaw member 511, e.g., to facilitate assembly and/or provide strength and rigidity. In alternative embodiments not shown, the inner surface of the structural insert 541 may include detents, tongue and groove interfaces, locking tabs, adhesive ports, etc., utilized either alone or in combination for assembly purposes.

In some embodiments, as shown in FIG. 5, the second portion 514 of the jaw member 511 defines a wall member 515 that extends outwardly of the outer lateral surface 536 of the third portion 516 and the outer lateral surface 532 of first portion 512, e.g., to provide strength and rigidity. Wall member 515 may have any suitable length “L₂”. In some embodiments, the cavity 521 and the wall member 515, as well as other features of the jaw member 511, may be formed by fineblanking. In some embodiments, the length “L₂” of the wall member 515 may be substantially equal to the length “L₁” of the structural insert 541.

Arm member 513 may define one or more apertures at least partially therethrough, e.g., pivot holes and/or pin slots or openings. In some embodiments, as shown in FIG. 5, the arm member 513 includes an elongated angled slot 581 and a pivot hole 586 defined therethrough. The shape, size and spacing of the slot 581 and the pivot hole 586 may be varied from the configuration depicted in FIG. 5. First arm member 513 may include additional, fewer, or different apertures than shown in FIG. 5. Jaw member 511 may additionally, or alternatively, include one or more jaw alignment spacers, e.g., jaw alignment spacer 575, integrally formed with or otherwise coupled to the arm member 513.

FIGS. 6 and 7 show a jaw assembly (shown generally as 600) according to an embodiment of the present disclosure that includes the jaw member 511 and the structural insert 541 shown in FIG. 5, an electrically-conductive tissue-engaging surface or sealing plate 660, and a non-electrically conductive member 639. Non-electrically conductive member 639 may be configured to support an electrically-conductive tissue-engaging surface or sealing plate 660 (FIG. 7) thereon. In some embodiments, as shown in FIG. 7, a bonding material 750, e.g., brazing material, adhesive material, or solder material, may be disposed between the sealing plate 660 and the non-electrically conductive member 639 and/or within the cavity 521, or portion thereof, between the structural insert 541 and the jaw member 511, e.g., to facilitate assembly and/or provide strength and rigidity.

Sealing plate 660 and the non-electrically conductive member 639 form a longitudinally-oriented slot or knife channel 615 defined therethrough for reciprocation of a knife blade (not shown). Support base 519 together with the non-electrically conductive member 639 may be encapsulated by the sealing plate 660 and/or an outer housing (not shown). Examples of sealing plate, outer housing, and knife blade embodiments are disclosed in commonly assigned International Application Ser. No. PCT/US01/11412 filed on Apr. 6, 2001, entitled “ELECTROSURGICAL INSTRUMENT WHICH REDUCES COLLATERAL DAMAGE TO ADJACENT TISSUE”, and commonly assigned International Application Ser. No. PCT/US01/11411 filed on Apr. 6, 2001, entitled “ELECTROSURGICAL INSTRUMENT REDUCING FLASHOVER”.

FIG. 8 shows an end view of an end-effector assembly (shown generally as 800) according to an embodiment of the present disclosure that includes the jaw assembly 600 shown in FIGS. 6 and 7 and an opposing jaw assembly 700. Second jaw assembly 700 generally includes an arm member 713. Second jaw assembly 700 is similar to the first jaw assembly 600 and further description thereof is omitted in the interests of brevity. End-effector assembly 800 includes a knife slot 815 defined by the arm members 613 and 713 for reciprocation of a knife blade 805.

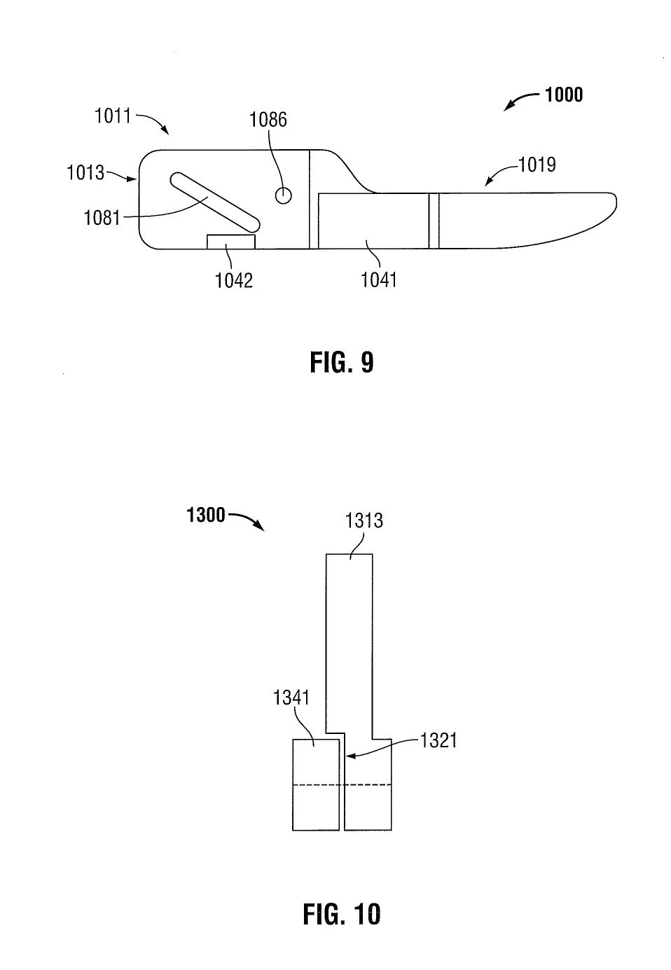

FIG. 9 shows a portion of a jaw assembly (shown generally as 1000) according to an embodiment of the present disclosure that includes a jaw member 1011 including an arm member 1013 and a support base 1019. Arm member 1013 includes an elongated angled slot 1081 and a pivot hole 1086 defined therethrough. In some embodiments, as shown in FIG. 9, the support base 1019 includes a structural insert 1041. Jaw member 1011 may additionally, or alternatively, include one or more jaw alignment spacers, e.g., jaw alignment spacer 1042, integrally formed with or otherwise coupled to the arm member 1013. Structural insert 1041 and the jaw alignment spacer 1042 shown in FIG. 9 are similar to the structural insert 541 and the jaw alignment spacer 575 shown in FIG. 5, and further description thereof is omitted in the interests of brevity.

FIGS. 10 and 11 show a portion of a first jaw assembly (shown generally as 1300) according to an embodiment of the present disclosure that includes a first jaw member 1311. First jaw member 1311 includes a first arm member 1313 and a first support base 1319 (FIG. 11). First jaw member 1311 may define one or more apertures at least partially therethrough, e.g., pivot holes and/or pin slots or openings. In some embodiments, as shown in FIG. 11, the first arm member 1313 includes an elongated angled slot 1381 and a pivot hole 1386 defined therethrough. In some embodiments, the first jaw assembly 1300 includes a structural insert 1341 associated with the first arm member 1313 and/or the first support base 1319.

As shown in FIG. 10, the first jaw assembly 1300 defines a cavity 1321 configured to receive at least a portion of the structural insert 1341 therein. In some embodiments, as shown in FIG. 11, first jaw member 1311 includes semi-pierce features 1315 associated with the horizontal cavity 1321 (FIG. 10). Structural insert 1341 is configured to be receivable (in whole or in part) within the cavity 1321.

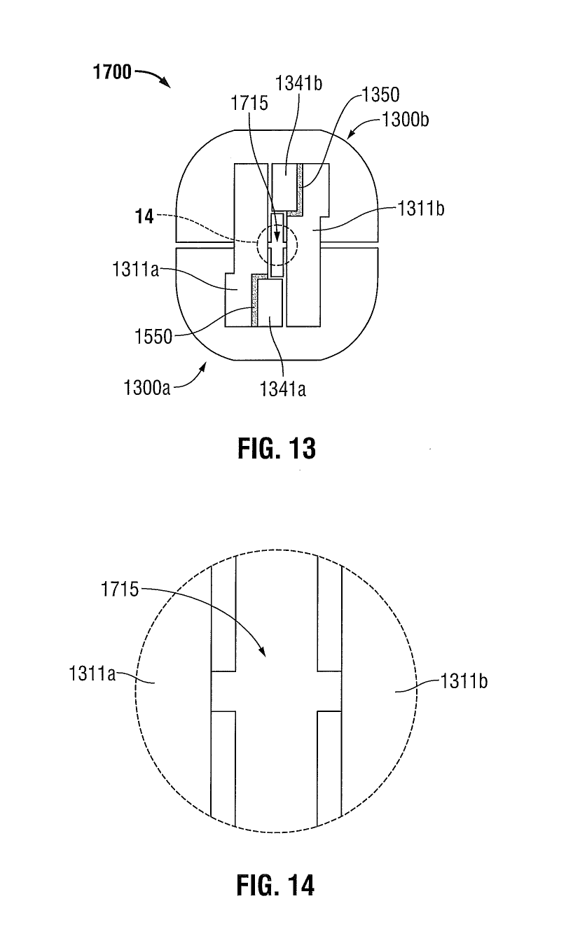

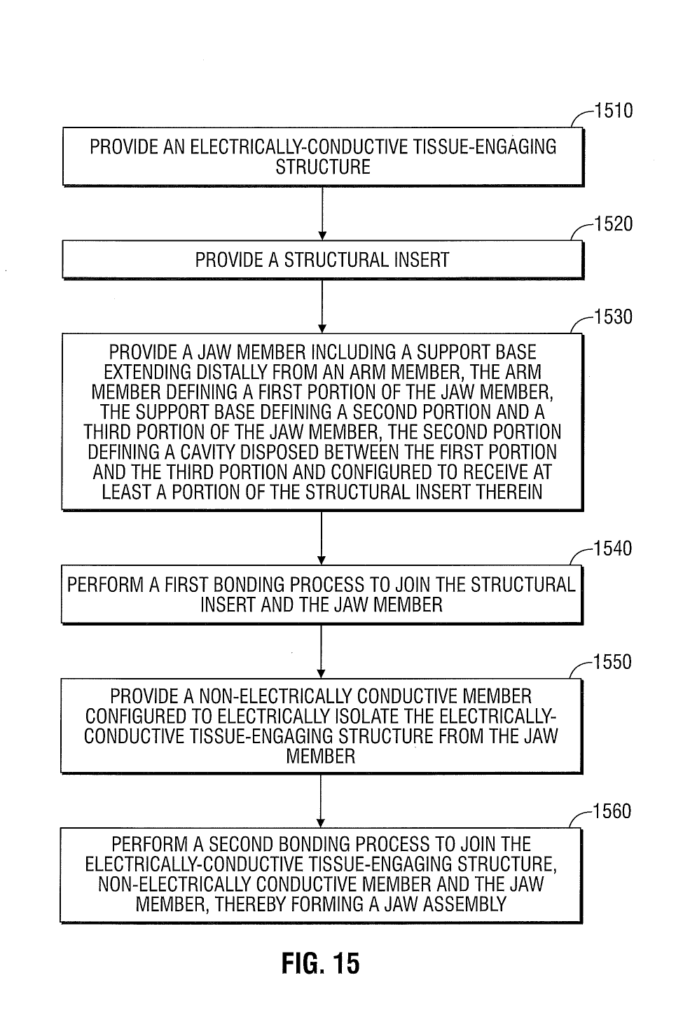

FIG. 12 shows a bottom view of the first jaw member 1311 and the structural insert 1341 shown in FIG. 11. Structural insert 1341 may be formed from any suitable material, and may be joined to the first jaw assembly 1300 by any suitable process, e.g., welding, brazing, soldering, and/or adhesive bonding. A bonding material 1350, e.g., brazing material, adhesive material, or solder material, may be disposed within the cavity 1321, or portion thereof, between the structural insert 1341 and the first jaw assembly 1300, e.g., to facilitate assembly and/or provide strength and rigidity. In alternative embodiments not shown, the inner surface of the structural insert 1341 may include detents, tongue and groove interfaces, locking tabs, adhesive ports, etc., utilized either alone or in combination for assembly purposes.

FIG. 13 shows a cross-sectional view of an end-effector assembly (shown generally as 1700) according to an embodiment of the present disclosure that includes a first jaw assembly 1300a and a second jaw assembly 1300b. First jaw assembly 1300a includes a first jaw member 1311a and a first structural insert 1341a associated therewith. Second jaw assembly 1300b includes a second jaw member 1311b and a second structural insert 1341b associated therewith. First and second jaw assemblies 1300a and 1300b, respectively, are similar to the first jaw assembly 1300 shown in a FIG. 11, and further description thereof is omitted in the interests of brevity. As shown in FIGS. 13 and 14, the first jaw assembly 1300a and the second jaw assemblies 1300a and 1300b, respectively, when assembled, form a knife channel 1715 defined therethrough for reciprocation of a knife blade (e.g., knife blade 805 shown in FIG. 8).

In alternative embodiments not shown, compatible with any of the above embodiments of jaw members for assembly into jaw assembly configurations, an electrically-insulative bushing may be used to electrically isolate the opposing jaw members from one another, wherein a configuration of one or more electrically-insulative bushings may be associated with either or both jaw members.

Hereinafter, a method of manufacturing a jaw assembly is described with reference to FIG. 15. It is to be understood that the steps of the method provided herein may be performed in combination and in a different order than presented herein without departing from the scope of the disclosure.

FIG. 15 is a flowchart illustrating a method of manufacturing a jaw assembly 600 according to an embodiment of the present disclosure. In step 1510, an electrically-conductive tissue-engaging structure 660 is provided.

In step 1520, a structural insert 541 is provided.

In step 1530, jaw member 511 including a support base 519 extending distally from an arm member 513 is provided. The arm member 513 defines a first portion 512 of the jaw member 511. The support base 519 defines a second portion 514 and a third portion 516 of the jaw member 511. The second portion 514 defines a cavity 521 disposed between the first portion 512 and the third portion 516 and configured to receive at least a portion of the structural insert 541 therein.

In some embodiments, the cavity 521 is disposed between the distal end 517 of the first portion 512 and the proximal end 518 of the third portion 516. Arm member 513 may define at least one aperture at least partially therethrough.

In step 1540, a first bonding process is performed to join the structural insert 541 and the jaw member 511. Structural insert 541 may be joined to the jaw member 511 by any suitable process, e.g., welding, brazing, soldering, and/or adhesive bonding. A bonding material 650, e.g., brazing material, adhesive material, or solder material, may be disposed within the cavity 521, or portion thereof.

In step 1550, a non-electrically conductive member 639 is provided. Non-electrically conductive member 639 is configured to electrically isolate the electrically-conductive tissue-engaging structure 660 from the jaw member 511.

In step 1560, a second bonding process is performed to join the electrically-conductive tissue-engaging structure 660, non-electrically conductive member 639 and the jaw member 511, thereby forming a jaw assembly 600. In some embodiments, a brazing process is performed to join the electrically-conductive tissue-engaging structure 660, non-electrically conductive member 639 and the jaw member 511. It will be appreciated that additional manufacturing steps may be undertaken after the step 1540, prior to the second bonding process in the step 1560.

Alternatively, in other embodiments, steps 1540, 1550 and 1560 may be combined into one step wherein the structural insert 541, the jaw member 511, the non-electrically conductive member 639, and the electrically-conductive tissue-engaging structure 660 are joined together, e.g., simultaneously, or in alternate orders via brazing or other suitable bonding processes.

The above-described bipolar forceps is capable of directing energy into tissue, and may be suitable for use in a variety of procedures and operations. The above-described end-effector embodiments may utilize both mechanical clamping action and electrical energy to effect hemostasis by heating tissue and blood vessels to coagulate, cauterize, cut and/or seal tissue. The jaw assemblies may be either unilateral or bilateral. The above-described bipolar forceps embodiments may be suitable for utilization with endoscopic surgical procedures and/or hand-assisted, endoscopic and laparoscopic surgical procedures. The above-described bipolar forceps embodiments may be suitable for utilization in open surgical applications.

The above-described method of manufacturing a jaw assembly may result in the formation of jaw assemblies that meet specific tolerance requirements for proper jaw alignment and other tightly-toleranced jaw assembly features. The above-described method of manufacturing a jaw assembly may provide improved thermal resistance, strength and rigidity of jaw assemblies.

Although embodiments have been described in detail with reference to the accompanying drawings for the purpose of illustration and description, it is to be understood that the inventive processes and apparatus are not to be construed as limited thereby. It will be apparent to those of ordinary skill in the art that various modifications to the foregoing embodiments may be made without departing from the scope of the disclosure.

What is claimed is:

1. A jaw assembly, comprising:
a jaw member, including:
an arm member defining a first portion of the jaw member; and
a support base extending distally from the arm member and defining a second portion and a third portion of the jaw member, wherein the second portion defines a cavity disposed between the first portion and the third portion; and
a structural insert, wherein at least a portion of the structural insert is disposed within the cavity.

2. The jaw assembly of claim 1, wherein the first portion includes a wall member that extends outwardly of an outer lateral surface of the third portion of the jaw member.

3. The jaw assembly of claim 1, wherein the wall member extends outwardly of an outer lateral surface of first portion of the jaw member.

4. The jaw assembly of claim 1, wherein a length of the structural insert is substantially equal to a length of the wall member.

5. The jaw assembly of claim 1, further comprising an electrically-conductive tissue-engaging structure.

6. The jaw assembly of claim 5, further comprising a non-electrically conductive member configured to electrically isolate the electrically-conductive tissue-engaging structure from the support base of the jaw member.

7. The jaw assembly of claim 6, wherein the electrically-conductive tissue-engaging structure and the non-electrically conductive member cooperatively define a longitudinally-oriented knife channel therethrough.

8. An end-effector assembly, comprising:
opposing first and second jaw assemblies pivotably mounted with respect to one another, wherein the first jaw assembly includes a first jaw member and the second jaw assembly includes a second jaw member;
the first jaw member including:
a first arm member defining at least one aperture at least partially therethrough and defining a first portion of the first jaw member;
a first support base extending distally from the first arm member and defining a second portion and a third portion of the first jaw member, wherein the second portion defines a cavity disposed between the first portion and the third portion; and
a structural insert, wherein at least a portion of the structural insert is disposed within the cavity;
the second jaw member including:
a second arm member defining at least one aperture at least partially therethrough; and
a second support base extending distally from the second arm member;
the first jaw assembly further including:
a first electrically-conductive tissue-engaging structure; and
a first non-electrically conductive member configured to electrically isolate the electrically-conductive tissue-engaging structure from the first support base of the first jaw member;
the second jaw assembly further including:
a second electrically-conductive tissue-engaging structure; and
a second non-electrically conductive member configured to electrically isolate the electrically-conductive tissue-engaging structure from the second support base of the second jaw member; and
at least one pivot pin engaged with the at least one apertures of the first and second jaw members such that the first and second jaw assemblies are pivotably mounted with respect to one another.

9. The end-effector assembly of claim 8, wherein the first electrically-conductive tissue-engaging structure and the first non-electrically conductive member cooperatively define a longitudinally-oriented knife channel therethrough.

10. The end-effector assembly of claim 8, wherein the second jaw assembly is adapted to connect the second electrically-conductive tissue-engaging structure associated therewith to an electrosurgical generator.

11. A method of manufacturing a jaw assembly, comprising the steps of:
providing an electrically-conductive tissue-engaging structure;
providing a structural insert;
providing a jaw member including a support base extending distally from an arm member, the arm member defining a first portion of the jaw member, the support base defining a second portion and a third portion of the jaw member, the second portion defining a cavity disposed between the first portion and the third portion and configured to receive at least a portion of the structural insert therein;
performing a first bonding process to join the structural insert and the jaw member;
providing a non-electrically conductive member configured to electrically isolate the electrically-conductive tissue-engaging structure from the jaw member; and
performing a second bonding process to join the electrically-conductive tissue-engaging structure, non-electrically conductive member and the jaw member, thereby forming a jaw assembly.

12. The method of manufacturing a jaw assembly of claim 11, wherein the cavity is disposed between a distal end of the first portion and a proximal end of the third portion.
